(19) United States
(12) Patent Application Publication
(10) Pub. No.: US 2024/0261157 A1
(43) Pub. Date: Aug. 8, 2024

(54) Men's Adult Diaper

(71) Applicant: Tony Vondell Drew, Detroit, MI (US)

(72) Inventor: Tony Vondell Drew, Detroit, MI (US)

(21) Appl. No.: 18/436,994

(22) Filed: Feb. 8, 2024

Related U.S. Application Data
(60) Provisional application No. 63/483,917, filed on Feb. 8, 2023.

(51) Int. Cl. A61F 13/491; A61F 13/49; A61F 13/495

(52) U.S. Cl. CPC A61F 13/4915; A61F 13/49009; A61F 13/495

(57) ABSTRACT

An adult male diaper characterized with a front pouch for urine and a rear pouch for feces. The diaper is to aid those who suffer from loss or bowel and bladder function. front pouch is designed to be equipped with a urinary pad that catches urine that is released form the body due to urinary incontinence. The urinary pouch is positioned on the front side of the diaper. The urinary pouch contains a pad that absorbs the urine and the pouch is designed to catch any urine not absorbed by the pouch. A fecal pouch is located on the rear side of the diaper and holds feces that are excreted from the body. There are wires that run from the run of the diaper through the crotch region and have clasps on the other end of the wire. The claps are to hold the bulk of the fecal pouch up.

### FIELD OF THE INVENTION

The present invention relates generally to diapers. More specifically, the present invention relates to adult diapers for men.

### BACKGROUND OF THE INVENTION

Diapers are needed for a wide variety of reasons. As people get older, it can be difficult to control one's bodily functions. With age, one may lose control over their bladder. Alternatively, one may lose function over their bowels. Age is not the only reason one may lose control of their bodily functions. Medical ailments and loss of muscle control are also a contributing factor. It can be an embarrassing situation if one were to soil themselves while out in public. To aid in this, many wear adult diapers to prevent soiling their garments.

However, often times disposable diapers are bulky and add extra weight to a person in their private region. Further, having to wear a bulky diaper can bring shame to the wearer. Additionally, having to change a diaper and secure the straps, while keeping the diaper positioned where the diaper belongs against the body can be a struggle. To combat this, the present invention is a pair of men's undergarments that has a removable pouch in the front and on the rear side to catch bodily waste. It is an object of the present invention to provide a comfortable, odor free, and convenient diaper for men.

### BRIEF DESCRIPTION OF THE DRAWINGS

FIG. 1 shows a left front prospective view of an embodiment of the present invention.

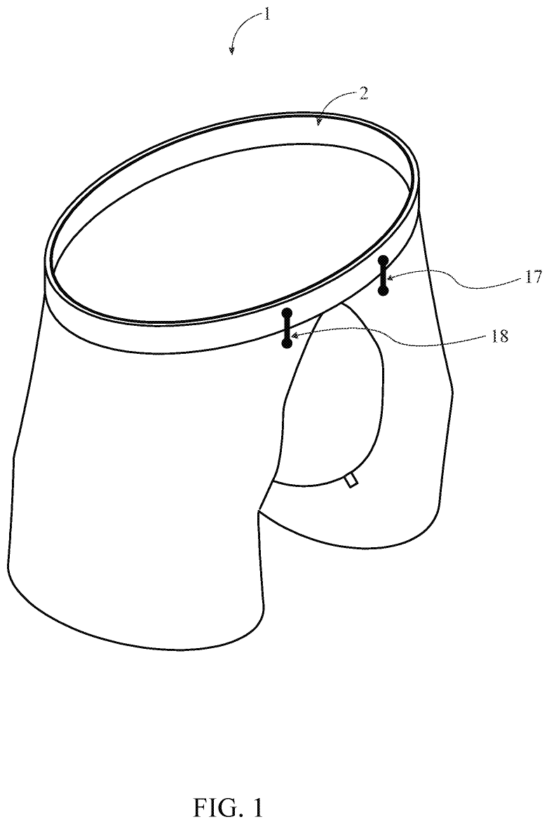

FIG. 2 shows a left rear prospective view of an embodiment of the present invention.

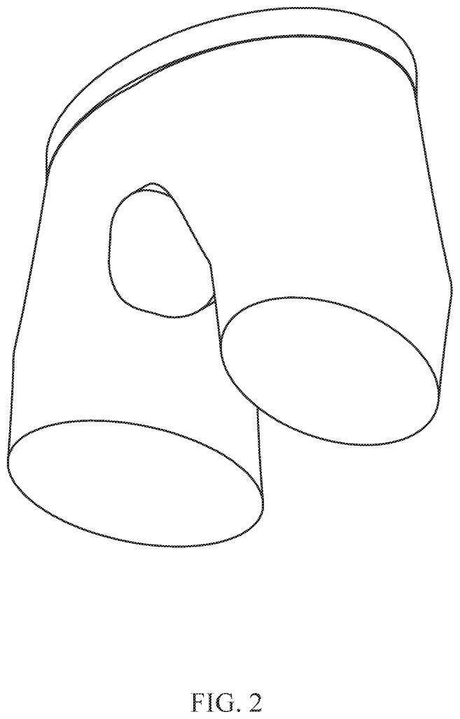

FIG. 3 shows a front elevational view of an embodiment of the present invention.

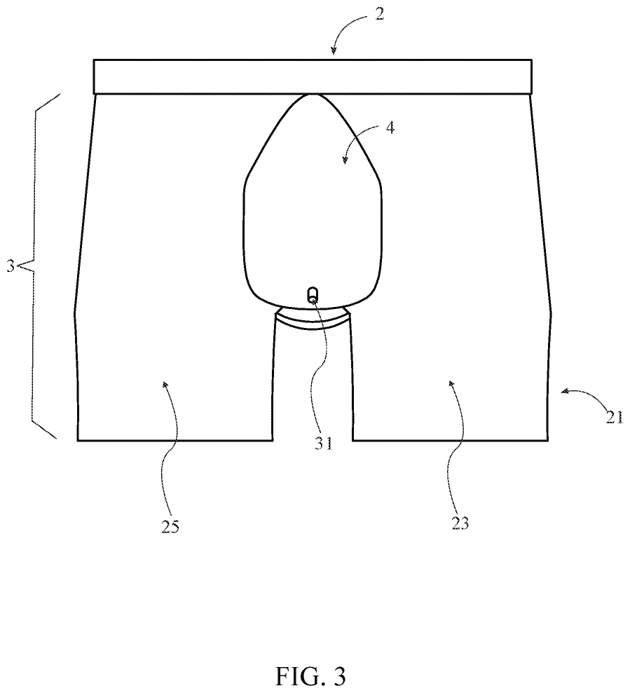

FIG. 4 shows a right elevational view of an embodiment of the present invention.

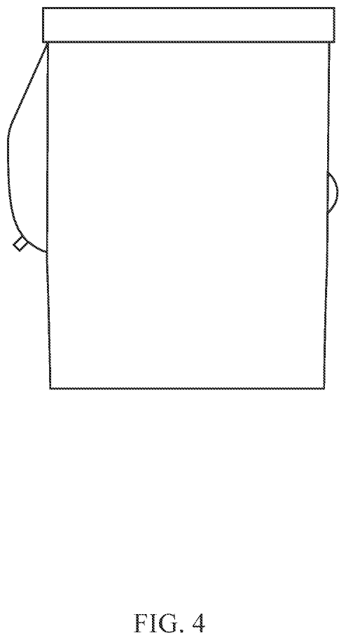

FIG. 5 shows a rear elevational view of an embodiment of the present invention.

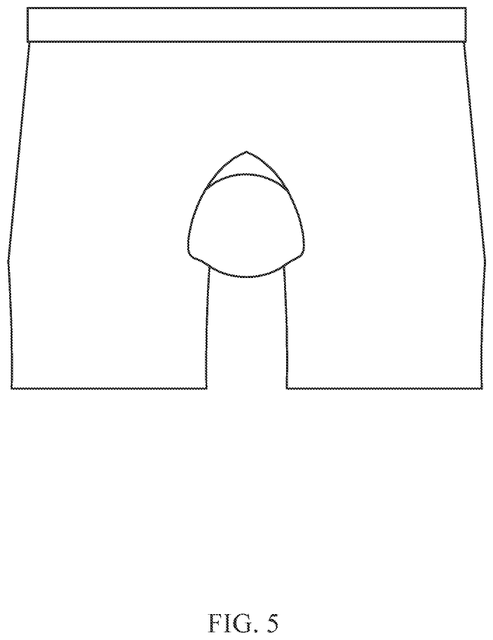

FIG. 6 shows a bottom plan view of an embodiment of the present invention.

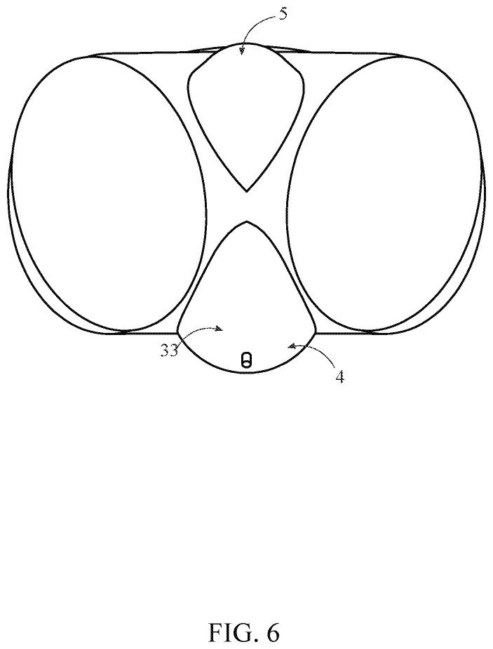

FIG. 7 shows a top plan view of an embodiment of the present invention.

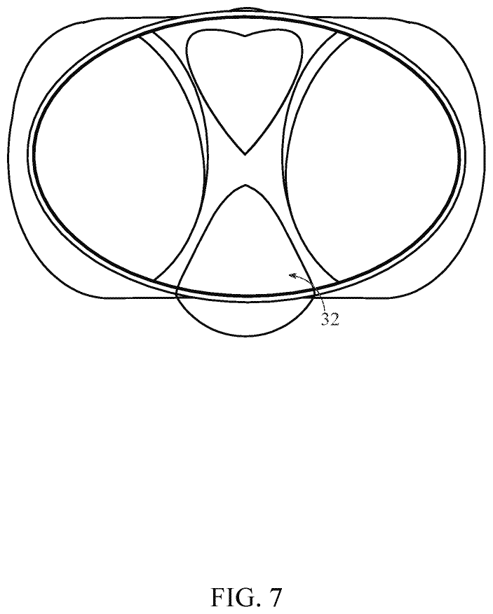

FIG. 8 shows a front elevational view of an embodiment of the present invention with the front urinary pouch removed.

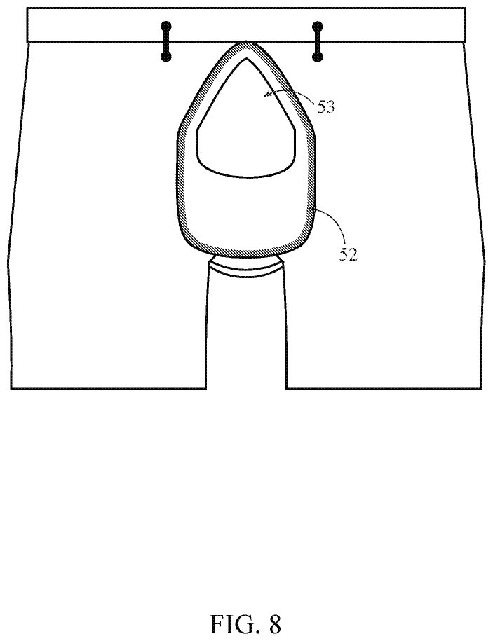

FIG. 9 shows a right front prospective view of an embodiment of the present invention with the front urinary pouch removed.

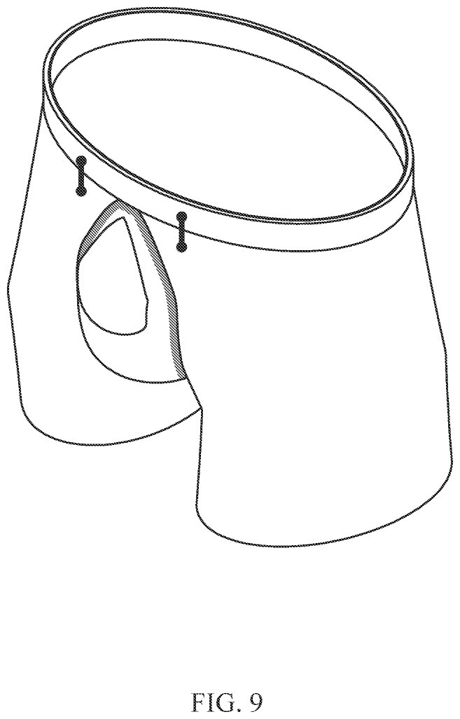

FIG. 10 shows a rear elevational view of an embodiment of the present invention with the fecal bag removed.

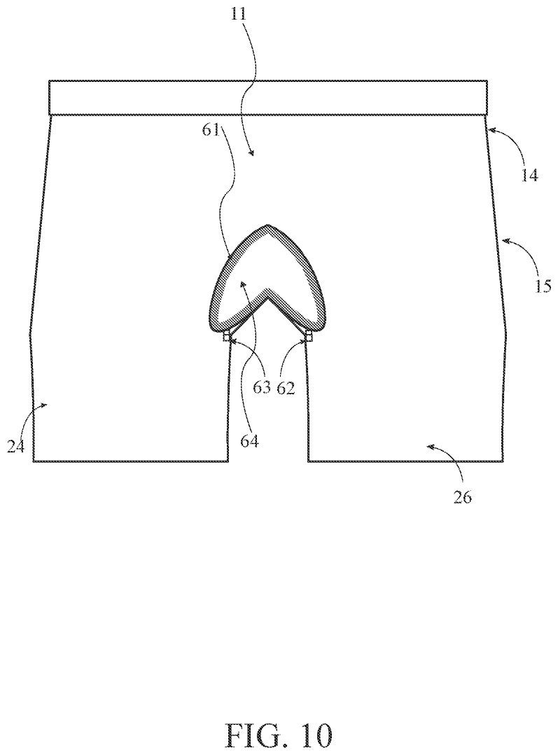

FIG. 11 shows a view of an embodiment of the present invention with the fecal bag in the released position.

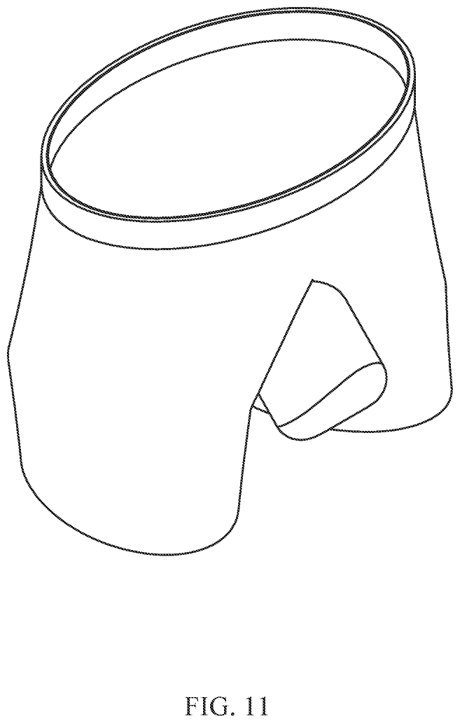

FIG. 12 shows the interior of the urinary pouch with the urinary pad in position.

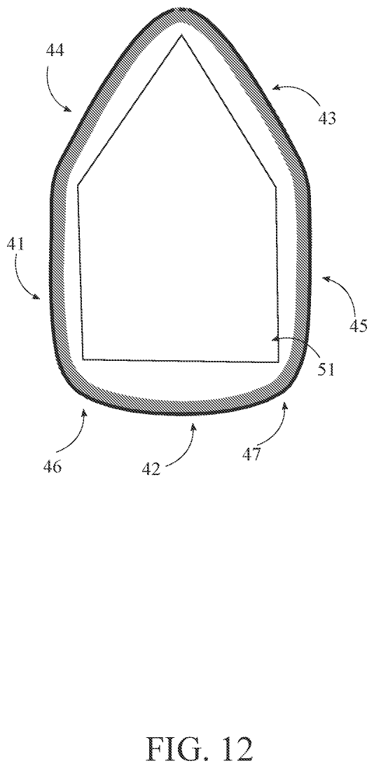

### DETAIL DESCRIPTIONS OF THE INVENTION

The present invention is an adult diaper 1 for men. This design removes the shame and embarrassment of wearing a diaper 1. It keeps the body dry, and bad odor free, while wearing and/or in the trash. It keeps feces off the body after a bowel movement. It makes it more convenient for the wearer to put on, take off and dispose of. This invention also have a back up plan, for emergencies. Example: if the wearer is away from home, and forgets to bring extra diaper 1 pads, and needs to change their pads; this invention solves the problem.

It works by separating the penis and the urinary pad 51 from the body, while being attached to the actual underwear, which is with a washable fabric material. The Poop Bag, touches the body, but separates the feces from the body, when the Emergency Release Clamps are released. The urinary pad 51 has an emergency urine drainage spout 31, located on the outside of the urinary pouch 4, near the bottom of the urinary pouch 4; this drainage spout 31 releases urine from the Diaper 1 Pouch in cases of emergency. The urinary pad 51 and fecal pouch 5 detaches from the fabric underwear for convenient disposal and clean up. Poly bag seals are used to seal the “DPP and Poop Bag” after use.

The components of this invention: washable fabric material, plastic(to form pouch and Poop bag)Bag, poly bag seals, thin plastic wire, plastic Clamps, washable Velcro, flexible rubber valve, scent blocker and liquid absorbent material.

The diaper 1 has an elastic waistband 2 and a body element 3. The body element 3 has an internal surface 12 and an external surface 13. The internal surface 12 is the surface that is in direct contact with the wearers body. The body element 3 comprises a front surface 10, a rear surface 11, an upper end 14 and a lower end 15. The elastic waistband 2 is perimetrically positioned around the upper end 14 of the body element 3. This is the same construction one would find in a standard pair of undergarments. Given that the diaper 1 is made out of fabric material, it is design to mimic that of regular undergarments. The body element 3 further comprises a left leg chute 21 and a right leg chute 22. The left leg chute and right leg chute are fused together at the lower end of the body element 3 forming the crotch region. The urinary aperture 53 is positioned on the front surface 10 of the body element 3 where the left leg chute 21 and the right leg chute 22 fuse to the lower end 15 of the body element 3. The urinary aperture 53 is the hole in which the urinary pouch 4 attaches to the diaper 1 by way of Velcro.

The “DPP” has poly bag seals and scent blockers around the opening of the pouch; the pouch is made of plastic, with the liquid absorbent material inside of it; the plastic pouch holds the urine while the absorbent material prevents the urine from going back out, from the way it came in.

The emergency drainage spout 31 is located on the outside, near the bottom of the urinary pouch 4. It's made of flexible rubber that retracts in and out; it's used to release urine from the urinary pad 51. The drainage spout 31 is positioned above the base edge 42 of the urinary pouch 4. The drainage spout 31 traverses from the internal surface of the urinary pouch 4 to the external surface of the urinary pouch 4. The drainage spout 31 has to traverse from inside the urinary pouch 4 to the outside to allow drainage of any accumulated urine that may not be absorbed by a urinary pad 51 that is placed within the urinary pouch 4. The drainage spout 31 opens and closes such that when closed, prevents leakage from the rubber valve.

The urinary pouch 4 comprising a urinary pad 51 and urinary Velcro 52. The urinary pouch 4 is disposable, but can be reused, in emergency situations. The urinary Velcro 52 is perimetrically positioned around the internal surface 32 urinary pouch 4. The urinary Velcro 52 is also positioned around the external surface 33 of the urinary aperture 53. This allows the Velcro on the urinary pouch 4 to “Velcro” to the diaper 1. The urinary pad 51 is smaller in size than the urinary pouch 4 allowing it to fit within the urinary pouch 4. The urinary pad 51 is positioned on the internal surface 32 of the urinary pouch 4 and runs from the pouch to in between the legs of the user. The urinary pouch 4 has a base edge 42, a left wall 41, a right wall 45, a first angled edge 43 and a second angled edge 44. The base edge 42 having a left end 46 and right end 47. The left edge 41 connects to the left end 46 of the base edge 42. The right edge 45 connects to the right end 47 of the base edge 42. The first angled edge 43 is positioned between the second angled edge 44 and the right edge 45. The second angled edge 44 being positioned between the first angled edge 46 and the left edge 41. The urinary pouch 4 having an external surface 33 and an internal surface 32. When these edges and walls connect, it forms a 5-sided hexagonal shaped pouch.

The fecal pouch 5 is positioned around the anus in order to catch feces. Poly bag seals and scent blockers are used convenient disposal of the fecal bag and to eliminate bad odor from the feces. The fecal has two thin plastic wires, a first wire and a second wire, which is routed from the front of the underwear to the bottom of the underwear; the plastic wire is connected to two plastic clamps; the plastic clamps holds the fecal bag in position. The underwear is made of fabric material, formed or stitched together to form men's briefs. The fecal pouch 5 has fecal Velcro 61, a left release clamp 63, and a right release clamp 62. The fecal pouch 5 has an external surface 33 and an internal surface 32. The fecal Velcro 61 is perimetrically positioned on the internal surface 32 of the fecal pouch 5. The fecal aperture 64 is positioned on the rear surface 11 of the body element 3 where the left leg chute 21 and the right leg chute 22 fuse to the lower end of the body element 3. The fecal pouch 5 is perimetrically attached to the fecal aperture 64.

The fecal pouch 5 is released when the left wire 17 and right wire 18 are pulled. When the plastic wires are pulled, the clamps open and the fecal bag drops downwards, but stays connected to the underwear by the Velcro. In short, the left release clamp 63 and the right release clamp 62 help hold up the bulk from the fecal pouch 5 but if the fecal pouch 5 needs to be dropped, the clamps can release the pouch. The left wire 17 is attached to the left release clamp 63 and the right wire 18 is attached to the right release clamp 62. The left release clamp 63 is secured to the fecal aperture 64 and the right release clamp 62 is secured to the fecal aperture 64. The elastic waistband 2 having a front face. The left wire 17 is sewn into the front face of the elastic waistband 2 and the right wire 18 is sewn into the front face of the elastic waistband 2. By having the wire sewn into the elastic waistband 2, it keeps the wire stationary and prevents it from prematurely dropping the fecal pouch 5. The left is being routed from the front face 70 of the elastic waistband 2 to the left release clamp 63 and the right wire 18 is routed from the front face of the elastic waistband 2 to the right release clamp 62.

Although the invention has been explained in relation to its preferred embodiment, it is to be understood that many other possible modifications and variations can be made without departing from the spirit and scope of the invention.

What is claimed is:

1. A men's adult diaper:
an elastic band
a body element;
the body element having an internal surface and an external surface
a left wire;
a right wire;
a power button;
a urine pouch;
a left release clamp and a right release clamp; and
a fecal pouch.

2. The men's adult diaper as claimed in claim 1, comprising:
the urinary pouch being perimetrically attached to the urinary aperture;
the urinary pouch comprising a base edge, a left wall, a right wall; a first angled edge and a second angled edge;
the base edge having a left end and right end;
the left edge connecting to the left end of the base edge;
the right edge connecting to the right end of the base edge;
the first angled edge being positioned between the second angled edge and the right edge;
the second angled edge being positioned between the first angled edge and the left edge; and
the urinary pouch having an external surface and an internal surface.

3. The men's adult diaper as claimed in claim 1, comprising:
the body element comprising a front surface, a rear surface, upper end and a lower end;
the elastic band being perimetrically positioned around the upper end of the body element;
the body element further comprising a left leg chute and a right leg chute;
the right leg chute and the left leg chute having an anterior surface and a posterior surface;
the left leg chute and the right leg chute each being fused to the lower end of the body element; and
a urinary aperture positioned on the front surface of the body element where the left leg chute and the right leg chute fuse to the lower end of the body element.

4. The men's diaper as claimed in claim 3 comprising:
the urinary pouch having a drainage spout;
the drainage spout being positioned above the base edge of the urinary pouch;
the drainage spout traversing from the internal surface of the urinary pouch to the external surface of the urinary pouch;
the urinary pouch comprising a urinary pad and urinary Velcro;
the urinary Velcro being perimetrically positioned around the internal surface urinary pouch;
the urinary pad being smaller in size than the urinary pouch; and
the urinary pad being centrally positioned on the internal surface of the urinary pouch.

5. The men's diaper as claimed in claim 1, comprising:
the feces pouch comprising fecal Velcro and a left release clamp and a right release clamp;
the fecal pouch having an external surface and an internal surface;
the fecal Velcro being perimetrically positioned internal surface of the fecal pouch;
a fecal aperture positioned on the rear surface of the body element where the left leg chute and the right leg chute fuse to the lower end of the body element; and
the fecal pouch being perimetrically attached to the fecal aperture.

6. The men's adult diaper as claimed in claim 5, comprising:
the left wire being attached to the left release clamp;
the right wire being attached to the right release clamp;
the left release clamp being secured to the fecal aperture; and
the right release clamp being secured to the fecal aperture.

7. The men's adult diaper as claimed in claim 1, comprising:
the elastic band having a front face;
the left wire being sewn into the front face of the elastic band;
the right wire being sewn into the front face of the elastic band;
the left wire being routed from the front face of the elastic band to the left release clamp; and
the right wire being routed from the front face of the elastic band to the right release clamp.

8. A men's adult diaper comprising:
an elastic band
a body element;
the body element having an internal surface and an external surface
a left wire;
a right wire;
a power button;
a urine pouch;
a left release clamp and a right release clamp; and
a fecal pouch;
the urinary pouch being perimetrically attached to the urinary aperture;
the urinary pouch comprising a base edge, a left wall, a right wall; a first angled edge and a second angled edge; and
the base edge having a left end and right end.

9. The men's adult diaper as claimed in claim 8 comprising:
the left edge connecting to the left end of the base edge;
the right edge connecting to the right end of the base edge;
the first angled edge being positioned between the second angled edge and the right edge;
the second angled edge being positioned between the first angled edge and the left edge; and
the urinary pouch having an external surface and an internal surface; the body element comprising a front surface, a rear surface, upper end and a lower end; and
the elastic band being perimetrically positioned around the upper end of the body element.

10. The men's adult diaper as claimed in claim 9 comprising:
the body element further comprising a left leg chute and a right leg chute;
the right leg chute and the left leg chute having an anterior surface and a posterior surface;
the left leg chute and the right leg chute each being fused to the lower end of the body element; and
a urinary aperture positioned on the front surface of the body element where the left leg chute and the right leg chute fuse to the lower end of the body element.

11. The men's adult diaper as claimed in claim 9 comprising:
the urinary pouch having a drainage spout;
the drainage spout being positioned above the base edge of the urinary pouch;
the drainage spout traversing from the internal surface of the urinary pouch to the external surface of the urinary pouch;
the urinary pouch comprising a urinary pad and urinary Velcro; and
the urinary Velcro being perimetrically positioned around the internal surface urinary pouch.

12. The men's adult diaper as claimed in claim 11 comprising:
the urinary pad being smaller in size than the urinary pouch; and
the urinary pad being centrally positioned on the internal surface of the urinary pouch;
the feces pouch comprising fecal Velcro and a left release clamp and a right release clamp;
the fecal pouch having an external surface and an internal surface;
the fecal Velcro being perimetrically positioned internal surface of the fecal pouch;
fecal aperture positioned on the rear surface of the body element where the left leg chute and the right leg chute fuse to the lower end of the body element;
the left release clamp being secured to the fecal aperture;
the right release clamp being secured to the fecal aperture; and
the fecal pouch being perimetrically attached to the fecal aperture.

13. The men's adult diaper as claimed in claim 8, comprising:
the left wire being attached to the left release clamp;
the right wire being attached to the right release clamp;
the elastic band having a front face;
the left wire being sewn into the front face of the elastic band;
the right wire being sewn into the front face of the elastic band;
the left wire being routed from the front face of the elastic band to the left release clamp; and
the right wire being routed from the front face of the elastic band to the right release clamp.
